(19) United States
(12) Patent Application Publication
(10) Pub. No.: US 2017/0028012 A1
(43) Pub. Date: Feb. 2, 2017

(54) Herbal Dietary Supplement for Increasing Testosterone and Improving Sexual Performance

(71) Applicant: Daniel Franz Faria, Smyrna, GA (US)

(72) Inventor: Daniel Franz Faria, Smyrna, GA (US)

(21) Appl. No.: 14/814,433

(22) Filed: Jul. 30, 2015

(51) Int. Cl. A61K 36/81; A61K 36/48; A61K 36/31; A61K 36/296; A23L 2/52; A61K 36/258; A61K 36/185; A61K 36/898; A61K 9/00; A23L 1/30; A61K 36/07; A61K 36/28

(52) U.S. Cl. CPC A61K 36/81; A61K 36/07; A61K 36/48; A61K 36/31; A61K 36/296; A61K 36/28; A61K 36/258; A61K 36/185; A61K 36/898; A61K 9/0095; A23L 1/3002; A23L 2/52; A23V 2002/00

(57) ABSTRACT

The present disclosure describes an herbal dietary supplement for increasing the testosterone level, libido, and sexual performance of a human. The herbal dietary supplement also increases the energy level of a human. The herbal dietary supplement may be prepared and provided in the forms of a liquid beverage or food bar for oral consumption. In such forms, the herbal dietary supplement comprises Ashwagandha and Mucuna Pruriens. Other ingredients may be included to further increase a human's testosterone level, libido, and/or energy level or to customize and/or enhance the effects of the other ingredients in the herbal dietary supplement. Such other ingredients may include, without limitation, Maca, Tinospora Cordiofolia, Asparagus Racemosus, Orchis Latifolia, Tribulus Terrestris, Anacylus Pyrethrum, L-dopa, Epimedium, King Oyster mushrooms, Vitamin-B6, Ginseng, and D-aspartic acid.

### FIELD OF THE INVENTION

The present invention relates, generally, to the field of dietary supplements for increasing testosterone levels in humans and improving sexual performance.

### BACKGROUND

Testosterone is an anabolic steroid hormone found in humans and various other mammals. In males, testosterone is produced and secreted primarily by the testicles. In females, testosterone is produced and secreted by the ovaries. Small amounts of testosterone are also produced and secreted by the adrenal glands in both males and females.

In human men, testosterone is the primary male sex hormone and plays a key role in the development of reproductive tissues such as the penis and scrotum, as well as promoting secondary sexual characteristics such as increased muscle and strength, bone mass and density, deepening of the voice, and the growth of body hair. Testosterone is also important to increasing libido and the frequency of erection. Additionally, testosterone is essential for health, well-being, and the prevention of osteoporosis.

Levels of testosterone in adult males are, typically, about seven to eight times greater than in adult females. As the metabolic consumption of testosterone in males is also greater, the daily production of testosterone is about twenty times greater in males. Testosterone levels decline gradually with age and testosterone levels may also be abnormally low due to causes such as testicular dysfunction or hypothalamic-pituitary dysfunction that is congenital or acquired. When testosterone levels are or become lower in males, the libido and frequency of erection may suffer. Additionally, muscle and bone mass may be lost. To counteract the body's lower production of testosterone and the adverse effects of lower testosterone levels, testosterone levels may be artificially increased through testosterone injections and transdermal patches and creams. While testosterone injections generally work well to increase testosterone levels, transdermal patches and creams have been less successful. Unfortunately, such injections must typically be administered by a health care professional, thereby requiring travel to and from the health care professional's office or other place of business. And, such injections, patches and creams may require a physician's prescription and be costly to obtain.

Therefore, there is a need for other products and methods of increasing testosterone levels in humans, and for resolving the difficulties, shortcomings, and problems of current products and methods described herein, in addition to other related or unrelated difficulties, shortcomings, and problems.

### SUMMARY OF THE INVENTION

Broadly described, the present invention comprises an herbal dietary supplement for increasing the level of testosterone in a human. The herbal dietary supplement also typically improves the libido, sexual performance, and energy level of a human who has taken the supplement. In example embodiments, the herbal dietary supplement may be in the forms of a liquid beverage or food bar for oral consumption. In such embodiments and forms, the herbal dietary supplement generally comprises Ashwagandha and Mucuna Pruriens. Other ingredients may be included in example embodiments to further increase a consumer's testosterone level, libido, and/or energy level or to customize and/or enhance the effects of the other ingredients in the herbal dietary supplement. Such other ingredients may include, but not be limited to, Maca, Tinospora Cordiofolia, Asparagus Racemosus, Orchis Latifolia, Tribulus Terrestris, Anacylus Pyrethrum, L-dopa, Epimedium, King Oyster mushrooms, Vitamin B6, Ginseng, and D-aspartic acid.

Advantageously, the herbal dietary supplement increases the consumer's testosterone level, libido, and sexual performance without the need for testosterone injections, transdermal patches or creams and without the need for a consumer to travel to a healthcare professional's office or other place of business. The herbal dietary supplement may be sold over the counter and taken absent a physician's prescription, thereby enabling the increase of testosterone level and libido at a cost lower than that of prescription drugs, injections, transdermal patches, and creams. As an added advantage or benefit, the herbal dietary supplement increases the consumer's energy level without the consumption of caffeine as is found in many of today's dietary supplements that claim to boost the consumer's energy level. Further, because the herbal dietary supplement may be embodied in the form of a non-refrigerated liquid beverage or food bar, the herbal dietary supplement is conveniently stored and transported, thereby permitting the herbal dietary supplement to be consumed at home, at work, in a vehicle, or at other locations.

Other uses, advantages and benefits of the present invention may become apparent upon reading and understanding the present specification.

### DETAILED DESCRIPTION OF EXAMPLE EMBODIMENTS

The herbal dietary supplement of the present invention, when consumed by a human, increases testosterone and energy levels and improves the libido and sexual performance. In accordance with the example embodiments described herein, the herbal dietary supplement may be prepared in the forms of a non-refrigerated liquid beverage or food bar, and comprises Ashwagandha and Mucuna Pruriens as primary active ingredients. The herbal dietary supplement may also, according the example embodiments, comprise secondary active ingredients, such as, but not limited to, Maca, Tinospora Cordiofolia, Asparagus Racemosus, Orchis Latifolia, Tribulus Terrestris, Anacylus Pyrethrum, L-dopa, Epimedium, King Oyster mushrooms, Vitamin B6, Ginseng, and D-aspartic acid. It should be understood and appreciated that in other example embodiments, the herbal dietary supplement may be prepared in other forms and may include the same or other ingredients in various amounts.

Ashwagandha (Withania somnifera) is a densely pubescent shrub that belongs to the Solanaceae family and grows up to one meter tall. The roots of this shrub include some alkaloids and vitanolids. The administration of Ashwagandha in the form of an aqueous extract increases the level of serum testosterone and LH (Luteinizing Hormone) levels in human males, while reducing the level of FSH (Follicle Stimulating Hormone). This result occurs, at least in part, due to the adaptogenic components of Ashwagandha reducing cortisol in the human body and, thereby, preserving testosterone molecules from oxidation.

Mucuna Pruriens is a leguminous plant member of the Fabaceae family. Mucuna Pruriens grows pods about twelve centimeters long that contain about seven seeds of varied coloration from beige to brown and black, or having stripes. Also known as velvet bean, lion bean, nescafe, and cowage, Mucuna Pruriens originated in India and is now grown in Sri Lanka, Malaysia, southeastern Asia, Brazil, and tropical regions of Central and South America. Studies indicate that Mucuna Pruriens has aphrodisiac properties, improves fertility and muscle mass, includes various sterols, and has high levels of L-dopa, an amino acid manufactured by the human body and a precursor to dopamine.

L-dopa is an amino acid and a hormone that is made naturally by a number of plants and animals. In humans, it is created via biosynthesis from the amino acid L-Tyrosine. It is a precursor to several important and powerful neurotransmitters which include dopamine, norepinephrine (noradrenaline) and epinephrine (adrenaline). These are collectively known as catecholamines and play a role in the stimulation and arousal systems in your brain, including stimulation and arousal related to sexual performance. In its pure form, L-dopa is considered to be a psychoactive chemical, and can also be used for the treatment of Parkinson's disease and a number of other conditions related to lowered levels of the above neurotransmitters (especially dopamine). As a nootropic, L-dopa is used to combat depression and anxiety while enhancing mood and improving the ability to concentrate and focus.

L-dopa causes a stimulatory effect on the human central nervous system resulting in the hypothalamus area of the brain, along with the pituitary gland, releasing increased levels of growth hormone (HGH). As a precursor, L-dopa has also been shown to increase the levels and activities of the adrenergic hormones norepinephrine and epinephrine. The combined presence of these increased levels of hormones and neurotransmitters are responsible for a number of beneficial effects like enhanced motor control, sex drive, immune function, fat loss (and gain), lean muscle growth, bone density, and energy level.

Maca (Lepidium meyenii walp) is a perennial tuber grown high in the Peruvian Andes. Despite poor soil conditions and a very harsh, arid environment, Maca thrives in the rarefied air above the 14,000 feet elevation at which it is grown. Maca is able to transfer the qualities of its hardy character to enhance endurance and stamina in humans and animals. With its high sterol profile, Maca is taken by bodybuilders as a potent alternative to anabolic steroids. Maca is also an aphrodisiac that increases vitality, libido, fertility and sexual performance and stamina. Additionally, Maca balances hormone levels, boosts energy, and fights impotence, stress, insomnia, and osteoporosis.

Tinospora Cordifolia, commonly referred to as “Guduchi”, belongs to the family Menispermaceae. Tinospora Cordifolia is native to India, found at higher altitude, and is a genetically diverse, large, deciduous climbing shrub with greenish yellow typical flowers. In racemes or racemose panicles, the male flowers are clustered and female are solitary. The flowering season expands over summers and winters. A variety of active components derived from the plant like alkaloids, steroids, diterpenoid lactones, aliphatics, and glycosides have been isolated from the different parts of the plant body, including root, stem, and whole plant. The plant has become of great interest to researchers across the globe because of its reported medicinal properties like anti-diabetic, anti-periodic, anti-spasmodic, anti-inflammatory, anti-arthritic, anti-oxidant, anti-allergic, anti-stress, anti-leprotic, anti-malarial, hepatoprotective, immunomodulatory and anti-neoplastic activities. By virtue of its anti-oxidant properties, Tinospora Cordiofolia contributes to less oxidation of testosterone molecules in the body, thereby increasing the level of testosterone present.

Asparagus Racemosus is a species of asparagus common throughout Nepal, Sri Lanka, India and the Himalayas. It grows one to two meters tall and prefers to take root in gravelly, rocky soils high up in piedmont plains, at 1,300 to 1,400 meters elevation. Asparagus Racemosus is a much branched spinus under-shrub with tuberous, short rootstock bearing numerous fusiform, succulent roots. It is mainly recommended as an aphrodisiac tonic and for rejuvenating the body. The male reproductive system benefits from Asparagus Racemosus as it increases anabolic activity in the body and reduces sexual debility, impotence, spermatorrhoea, and inflammation of sexual organs. It is believed that such benefits are due to lyophilized aqueous extracts obtained from the roots of Asparagus Racemosus that produce testosterone-like effects. Asparagus Racemosus is also used for upset stomach (dyspepsia), constipation, stomach spasms, and stomach ulcers. Additionally, it is used for fluid retention, pain, anxiety, cancer, diarrhea, bronchitis, tuberculosis, dementia, and diabetes.

Orchis Latifolia is a tuber that grows in the western Himalyas at an elevation of eight to twelve thousand feet. The stem is one to three feet in length with the apex part of the stem bearing leaves having a length of two to six inches. The flower stalk is one to six inches long and bears long purple flowers. The dried root of Orchis Latifolia has been long esteemed as great restorative and invigorative, and as an aphrodisiac. Orchis Latifolia also has astringent, cooling, demulcent, diuretic, emollient, expectorant, nutritive, retentative, sweet and tonic properties. By its action on the human nervous system, Orchis Latifolia helps prevent nervousness. Orchis Latifolia is also very useful in treating erectile dysfunction, seminal weakness, oligospermia, spermatorrhoea, premature ejaculation, nocturnal emissions, phthisis, diabetes, chronic diarrhoea, and dysentery.

Tribulus Terrestris is an annual plant in the Caltrop family (Zygophyllaceae) widely distributed around the world that is adapted to grow in dry climate locations in which few other plants can survive. It is native to warm temperate and tropical regions of southern Europe, southern Asia, throughout Africa, and Australia. A network of fine rootlets arise from the taproot to take advantage of soil moisture, enabling the plant to survive in very arid conditions of desert climates and in poor soil. Tribulus Terrestris is mostly recommended for male health including virility and vitality because it enhances libido and, purportedly, also boosts testosterone. While it is not exactly known how Tribulus Terrestris works, it is known to enhance androgen receptor density in the brain, which may enhance the libido enhancing properties of androgens.

Anacylus Pyrethrum is a perennial herb found in North Africa, elsewhere in the Mediterranean region, in the Himalayas, in North India, and in Arabian countries. The roots of Anacylus Pyrethrum improve anabolic activity in animals, increase testosterone levels, and enhance fertility and libido. It acts on the brain and nervous system to stimulate sexual desire and increase blood supply to the genitals.

Epimedium, also known as “barrenwort”, “bishop's hat”, “fairy wings”, “horny goat weed”, “rowdy lamb herb”, “randy beef grass” or “yin yang huo”, is a genus of flowering plants in the family Berberidaceae. The majority of the species are endemic to China, with smaller numbers elsewhere in Asia, and a few in the Mediterranean region. Epimedium species are deciduous or evergreen hardy perennials. The majority have four-parted “spider-like” flowers in spring. Herbal Epimedium contains icariin, which like sildenafil (the erectile dysfunction drug commonly sold as Viagra®), inhibits the activity of a phosphodiesterase type 5 (PDE-5) inhibitor. Epimedium is, therefore, used as an aphrodisiac and a treatment for erectile dysfunction.

Ginseng is any one of the eleven species of slow-growing perennial plants with fleshy roots, belonging to the genus Panax of the family Araliaceae. Ginseng is an herb having a light-colored, forked-shaped root, a relatively long stalk, and green leaves with an oval shape. Ginseng is found in North America and in eastern Asia (mostly Korea, northeast China, Bhutan, eastern Siberia), typically in cooler climates. Panax Ginsengs are adaptogenic herbs and are characterized by the presence of ginsenosides and gintonin. Both American Ginseng (Panax quinquefolius, L.) and Asian Ginseng (P. Ginseng) are believed to provide an energy boost, lower blood sugar and cholesterol levels, reduce stress, promote relaxation, treat diabetes, and treat erectile dysfunction in men.

King Oyster mushrooms (Pleurotus eryngii) are a species of the Pleurotus family of mushrooms, and related to regular Oyster mushrooms. King Oyster mushrooms are edible mushrooms native to Mediterranean regions of Europe, the Middle East, and North Africa, but also grown in many parts of Asia King Oyster mushrooms have a thick, white stem that confers a relatively “meaty” or “hearty” flavor (Umami), a small tan cap, and are considered to be a medicinal mushroom. Through interaction with the human immune system, King Oyster mushrooms may boost immunity to sickness. King Oyster mushrooms also seem to be potent at suppressing fatty acid absorption relative to other mushrooms, and have been implicated in increasing testosterone 5-fold.

Vitamin B6, also known as pyridoxine, is one of the B complex vitamins and is found in certain foods such as cereals, beans, vegetables, liver, meat, and eggs. Vitamin B6 is needed for normal brain and nervous system development and aids in the transmission of signals between nerve cells. It also helps the body make the hormones serotonin and norepinephrine, which influence mood, and melatonin, which helps regulate the body clock. At least by contributing to the ability of the human body to transmit signals between nerve cells and by improving mood, Vitamin B6 enhances sexual arousal and sexual performance.

D-aspartic acid is an endogenous amino acid present in neuroendocrine tissues of invertebrates and vertebrates, including humans. D-aspartic acid occurs principally in the pituitary gland and testes, and has a role in the regulation of the release and synthesis of LH (luteinizing hormone) and testosterone in humans. In men, a lower D-aspartic acid content was found in oligoasthenoteratospermic seminal fluid and spermatozoon, and a relationship exists between the amount and motility of semen and the content of D-aspartic acid. In women, it has been found that D-aspartic acid occurs in the follicular fluid as a physiological component, and that the concentration of D-aspartic acid in the fluid is reduced in older women. In addition, the concentration of D-aspartic acid in the follicular fluid is lower, as are the quality of the oocytes and the level of fertilization. It has also been found that sodium D-aspartic acid induces an enhancement of LH and testosterone release.

In the example embodiments, the herbal dietary supplement comprises plant ingredients from the plants described above in the form of plant extracts or derivatives thereof. A plant extract may be taken from the leaves, root, seeds or any other component of the plant, as appropriate, to provide maximum therapeutic effect. Any one of the plant extracts or derivatives thereof may be substituted with an active constituent that is believed to contribute to the improvement or enhancement of testosterone levels, energy levels, or sexual function or performance.

According to the example embodiments, the herbal dietary supplement comprises the ingredients and amounts listed in Table 1 below, including the foregoing plant extracts or derivatives thereof, Vitamin B6, and D-aspartic acid:

TABLE 1
Ingredient List

| | Amount | | Preferred | | Preferred | |
|---|---|---|---|---|---|---|
| Ingredient | Range | | Range | | Amount | |
| Ashwaghanda | 0-3,200 | mg | 1,200-2,000 | mg | 1,600 | mg |
| Mucuna Pruriens | 0-1,500 | mg | 150-900 | mg | 500 | mg |
| Maca | 0-1,500 | mg | 250-750 | mg | 500 | mg |
| Tinospora Cordifolia | 0-20 | g | 10-15 | g | 12 | g |
| Asparagus Racemosus | 0-4,500 | mg | 1,100-3,600 | mg | 2,000 | mg |
| Orchis Latifolia | 0-10 | g | 3-5 | g | 4 | g |
| Tribulus Terrestris | 0-150 | mg | 55-90 | mg | 75 | mg |
| Anacylus Pyrethrum | 0-3,000 | mg | 500-2,000 | mg | 1,250 | mg |
| Epimedium | 0-2,750 | mg | 900-1,500 | mg | 1,200 | mg |
| Ginseng | 0-3,500 | mg | 600-2,400 | mg | 1,800 | mg |
| King Oyster Mushrooms | 0-7.5 | g | 1-3 | g | 2 | g |
| Vitamin B6 | 0-5 | mg | 1.3-1.7 | mg | 1.5 | mg |
| D-aspartic Acid | 0-4,500 | mg | 2,000-3,000 | mg | 2,500 | mg |

In a first example embodiment, the herbal dietary supplement comprises a liquid beverage for oral consumption one time per day and includes the ingredients identified in Table 1 in their respective preferred amounts. The ingredients are combined, for example and not limitation, with fruit, fruit juice, vegetables, vegetable juice, water, milk, soy, sugar, sweetener, food oils, flavoring agents, dyes, coloring agents, excipients, preservatives, and/or other therapeutically inactive ingredients as appropriate or desired, to produce a tasteful liquid beverage. It should be understood and appreciated that other amounts of the ingredients identified in Table 1 may be combined in a liquid beverage for consumption multiple times per day. It should also be understood and appreciated that a liquid beverage form of the herbal dietary supplement may include some or all of the ingredients identified in Table 1.

In a second example embodiment, the herbal dietary supplement comprises a food bar (such as, for example, a snack bar) for oral consumption one time per day and includes the ingredients identified in Table 1 in their respective preferred amounts. The ingredients are combined, for example and not limitation, with fruit, fruit juice, vegetables, vegetable juice, water, milk, soy, sugar, sweetener, food oils, binders, flavoring agents, dyes, coloring agents, excipients, preservatives, and/or other therapeutically inactive ingredients as appropriate or desired. Additionally, other ingredients may include, without limitation: oats, bran, granola, barley, rye, wheat, rice, maize, and other grains; peas, beans, lentils, soybeans, and other legumes; and/or chocolate, peanut butter, and other ingredients. Generally, the ingredients are ground (for ingredients in solid form) into a powder form, and are mixed or blended in a moistened state before being formed into food bars that may be baked or processed further to produce a tasteful food bar.

In another example embodiment, a method for treating low testosterone level and sexual dysfunction is provided. The method comprises administering the herbal dietary supplement described herein to a human in one or more forms.

Whereas the present invention has been described in detail above with respect to an example embodiment thereof, it should be appreciated that variations and modifications might be effected within the spirit and scope of the present invention.

What is claimed is:

1. An herbal dietary supplement for increasing the level of testosterone and improving sexual performance of a human, said herbal dietary supplement comprising:
Ashwaghanda; and
Mucuna Pruriens.

2. The herbal dietary supplement of claim 1, wherein said herbal dietary supplement comprises Ashwaghanda in an amount by weight between 0 and 3,200 milligrams.

3. The herbal dietary supplement of claim 2, wherein said herbal dietary supplement comprises Mucuna Pruriens in an amount by weight between 0 and 1,500 milligrams.

4. The herbal dietary supplement of claim 3, wherein said herbal dietary supplement comprises Ashwaghanda in an amount by weight of 1,600 milligrams.

5. The herbal dietary supplement of claim 4, wherein said herbal dietary supplement comprises Mucuna Pruriens in an amount by weight of 500 milligrams.

6. The herbal dietary supplement of claim 1, wherein said herbal dietary supplement further comprises Maca.

7. The herbal dietary supplement of claim 6, wherein said herbal dietary supplement comprises Maca in an amount by weight between 0 and 1,500 milligrams.

8. The herbal dietary supplement of claim 7, wherein said herbal dietary supplement comprises Maca in an amount by weight of 500 milligrams.

9. The herbal dietary supplement of claim 6, wherein said herbal dietary supplement further comprises King Oyster Mushrooms.

10. The herbal dietary supplement of claim 9, wherein said herbal dietary supplement comprises King Oyster Mushrooms in an amount by weight between 0 and 7.5 grams.

11. The herbal dietary supplement of claim 10, wherein said herbal dietary supplement comprises King Oyster Mushrooms in an amount by weight of 2 grams.

12. The herbal dietary supplement of claim 1, wherein said herbal dietary supplement is in the form of a liquid beverage.

13. The herbal dietary supplement of claim 1, wherein said herbal dietary supplement is in the form of a food bar.

14. An herbal dietary supplement for increasing the level of testosterone and improving sexual performance of a human, said herbal dietary supplement comprising:
Ashwaghanda;
Mucuna Pruriens; and
Maca.

15. The herbal dietary supplement of claim 14, said herbal dietary supplement further comprising Epimedium.

16. The herbal dietary supplement of claim 15, said herbal dietary supplement further comprising Anacylus Pyrethrum.

17. The herbal dietary supplement of claim 16, said herbal dietary supplement further comprising Ginseng.

18. The herbal dietary supplement of claim 17, said herbal dietary supplement further comprising Tribulus Terrestris.

19. The herbal dietary supplement of claim 18, said herbal dietary supplement further comprising Orchis Latifolia.

20. The herbal dietary supplement of claim 19, said herbal dietary supplement further comprising King Oyster Mushrooms.
